(19) United States
(12) Patent Application Publication
(10) Pub. No.: US 2023/0313178 A1
(43) Pub. Date: Oct. 5, 2023

(54) LIBRARY PREPARATION SYSTEMS AND METHODS

(71) Applicant: ILLUMINA, INC., San Diego, CA (US)

(72) Inventor: Bryan Crane, San Diego, CA (US)

(21) Appl. No.: 18/126,910

(22) Filed: Mar. 27, 2023

Related U.S. Application Data
(60) Provisional application No. 63/325,049, filed on Mar. 29, 2022.

(51) Int. Cl. C12N 15/10; B01L 3/00; B01L 7/00

(52) U.S. Cl. CPC C12N 15/1068; B01L 3/50851; B01L 7/52; B01L 2200/0668; B01L 2400/043; B01L 2200/16; B01L 2300/1805

(57) ABSTRACT

Library preparation systems and methods are disclosed. An apparatus includes a plate receptacle, a magnet, a thermocycler, and an actuator. The plate receptacle is to receive a plate having a well and the thermocycler is to adjust a temperature of a sample within the well of the plate and the actuator is to move the magnet relative to the plate receptacle.

### RELATED APPLICATION

This application claims the benefit of and priority to U.S. Provisional Patent Application No. 63/325,049, filed Mar. 29, 2022, the content of which is incorporated by reference herein in its entirety and for all purposes.

### BACKGROUND

DNA libraries may be prepared to allow samples to be sequenced.

### SUMMARY

Shortcomings of the prior art can be overcome and benefits as described later in this disclosure can be achieved through the provision of library preparation systems and methods. Various implementations of the apparatus and methods are described below, and the apparatus and methods, including and excluding the additional implementations enumerated below, in any combination (provided these combinations are not inconsistent), may overcome these shortcomings and achieve the benefits described herein.

In a first implementation, an apparatus includes a plate receptacle, a magnet, a thermocycler, and an actuator. The plate receptacle includes a thermal block and an insert. The thermocycler is in heat communication with the plate receptacle. The plate receptacle is to receive a plate having a well and the thermocycler is to adjust a temperature of a sample within the well of the plate. The actuator is to move the magnet relative to the plate receptacle and the insert is operable to transfer a magnetic field from the magnet to the plate receptable.

In a second implementation, a method includes adjusting a temperature of a sample within a well of a plate disposed in a plate receptacle including a thermal block and an insert using a thermocycler and dispensing beads into the well of the plate and a first reagent into the well of the plate with the plate disposed in the plate receptacle. The method includes moving the magnet toward the insert to allow the insert to transfer a magnetic field from the magnet to the plate receptable and draw the beads toward the magnet and aspirating the first reagent from the well with the plate disposed in the plate receptacle.

In a third implementation, an apparatus includes a plate receptacle, a magnet, a thermocycler, and an actuator. The plate receptacle is to receive a plate having a well and the thermocycler is to adjust a temperature of a sample within the well of the plate and the actuator is to move the magnet relative to the plate receptacle.

In further accordance with the foregoing the first, second, and/or third implementations, an apparatus and/or method may further include or comprise any one or more of the following:

In an implementation, the plate receptacle includes well receptacles and the insert is positioned within the thermal block.

In another implementation, the insert at least partially defines the well receptacles.

In another implementation, the insert includes at least one of a ferrous material, Nickel, a mu-metal, or a Cobalt alloy.

In another implementation, the insert extends horizontally through the thermal block.

In another implementation, the apparatus includes a heat sink coupled to the thermocycler.

In another implementation, the heat sink includes a liquid cooled heat sink.

In another implementation, the apparatus includes opposing first and second brackets positioned on either side of the plate receptacle. The magnet includes a first magnet coupled to the first bracket and a second magnet coupled to the second bracket and the actuator includes a first actuator that moves the first bracket and a second actuator that moves the second bracket relative to the plate receptacle to allow the first magnet and the second magnet to act on the plate receptacle.

In another implementation, the thermocycler includes a first thermoelectric cooler positioned on a first side of the plate receptacle and a second thermoelectric cooler positioned on a second side of the plate receptacle.

In another implementation, a heatsink is coupled to each of the first thermoelectric cooler and the second thermoelectric cooler.

In another implementation, the magnet includes a switchable magnet.

In another implementation, the actuator is to actuate the switchable magnet.

In another implementation, the plate receptacle includes a plurality of well receptacles and the insert includes a plurality of cupped pins. Each cupped pin is positioned beneath one of the well receptacles.

In another implementation, the apparatus includes a U-shaped bracket carrying the magnet and having ends and the insert includes a magnetic permeability material extending from the thermal block. The thermal block and the magnetic permeability material defining well receptacles.

In another implementation, the magnetic permeability material includes at least one of a ferrous material, Nickel, a mu-metal, or a Cobalt alloy.

In another implementation, the thermocycler is positioned within the U-shaped bracket.

In another implementation, the magnet includes a first magnet and a second magnet. The apparatus includes a first bracket carrying the first magnet and having first and second ends and a second bracket carrying the second magnet and having first and second ends. The first bracket and the second bracket opposing one another. The insert includes a first magnetic permeability material extending from the thermal block and a second magnetic permeability material extending from the thermal block. The thermal block, the first magnetic permeability material, and the second magnetic permeability material defining well receptacles.

In another implementation, the actuator moves the first bracket and the second bracket to allow the first magnetic permeability material to be coupled to and extend between the first ends of the first bracket and the second bracket and to allow the second magnetic permeability material to be coupled to and extend between the second ends of the first bracket and the second bracket.

In another implementation, the magnet includes a first magnet and a second magnet. The apparatus includes a first U-shaped bracket carrying the first magnet and having ends and a second U-shaped bracket carrying the second magnet and having ends. The insert includes a first magnetic permeability material extending from the thermal block and a second magnetic permeability material extending from the thermal block. The thermal block, the first magnetic permeability material, and the second magnetic permeability material defining well receptacles.

In another implementation, the actuator includes a first actuator and a second actuator. The first actuator to move the first U-shaped bracket to allow the first magnetic permeability material to be coupled to and extend between the ends of the first U-shaped bracket. The second actuator is to move the second U-shaped bracket to allow the second magnetic permeability material to be coupled to and extend between the ends of the second U-shaped bracket.

In another implementation, the apparatus includes a stage includes the thermocycler including the thermal block defining first well receptacles and the magnet includes second well receptacles spaced from the first well receptacles.

In another implementation, the magnet includes a plurality of ring magnets and each ring magnet surrounds one of the second well receptacles.

In another implementation, the method includes running a series of temperature adjustments using the thermocycler.

In another implementation, the method includes moving the magnet away from the insert.

In another implementation, the method includes dispensing a second reagent into the well of the first plate.

In another implementation, the method includes moving the magnet toward the insert to allow the insert to transfer a magnetic field from the magnet to the plate receptable and draw the beads toward the magnet.

In another implementation, the method includes aspirating the second reagent and the sample from the well of the plate.

In another implementation, the method includes dispensing the second reagent and the sample into a well of a second plate.

In another implementation, the thermocycler is to run a series of temperature adjustments.

In another implementation, beads are to be dispensed into the well of the plate and a first reagent is to be dispensed into the well of the plate.

In another implementation, the actuator is to move the magnet toward the plate receptacle to draw the beads toward the magnet and the first reagent is to be aspirated from the well.

In another implementation, a second reagent is to be dispensed into the well of the first plate.

In another implementation, the actuator is to move the magnet toward the plate receptacle to draw the beads toward the magnet, the second reagent and the sample are aspirated from the well of the plate, and the second reagent and the sample are dispensed into a well of a second plate.

In another implementation, the thermocycler is positioned beneath the plate receptacle.

In another implementation, the plate receptacle has a thermal block defining well receptacles and the thermocycler is positioned beneath the well receptacles.

In another implementation, the apparatus includes a heat sink coupled to the thermocycler.

In another implementation, the apparatus includes opposing first and second brackets positioned on either side of the plate receptacle. The magnet has a first magnet coupled to the first bracket and a second magnet coupled to the second bracket and the actuator includes a first actuator that moves the first bracket and a second actuator that moves the second bracket relative to the plate receptacle to allow the first magnet and the second magnet to act on the plate receptacle.

In another implementation, the thermocycler has a first thermoelectric cooler positioned on a first side of the plate receptacle and a second thermoelectric cooler positioned on a second side of the plate receptacle.

In another implementation, the apparatus includes a heatsink coupled to each of the first thermoelectric cooler and the second thermoelectric cooler.

In another implementation, the plate receptacle has a thermal block and a ferrous material insert in the thermal block.

In another implementation, the actuator moves the bracket and the magnet relative to the plate receptacle to allow the magnet to act on the plate receptacle.

In another implementation, the magnet has a switchable magnet.

In another implementation, the actuator is to actuate the switchable magnet.

In another implementation, the plate receptacle has a plurality of well receptacles and a plurality of cupped pins. Each cupped pin is positioned beneath one of the well receptacles.

In another implementation, the apparatus includes a U-shaped bracket carrying the magnet and having ends and the plate receptacle has a thermal block and a magnetic permeability material extending from the thermal block. The thermal block and the magnetic permeability material defining well receptacles.

In another implementation, the actuator moves the U-shaped bracket relative to the magnetic permeability material to allow the magnet to act on the magnetic permeability material.

In another implementation, the thermocycler is positioned within the U-shaped bracket.

In another implementation, the magnetic permeability material includes mu-metal.

In another implementation, the magnet has a first magnet and a second magnet. The apparatus also includes a first bracket carrying the first magnet and having first and second ends and a second bracket carrying the second magnet and having first and second ends. The first bracket and the second bracket oppose one another. The plate receptacle has a thermal block and a first magnetic permeability material extending from the thermal block and a second magnetic permeability material extending from the thermal block. The thermal block, the first magnetic permeability material, and the second magnetic permeability material defining well receptacles.

In another implementation, the actuator moves the first bracket and the second bracket to allow the first magnetic permeability material to be coupled to and extend between the first ends of the first bracket and the second bracket and to allow the second magnetic permeability material to be coupled to and extend between the second ends of the first bracket and the second bracket.

In another implementation, the thermocycler is positioned between the first bracket and the second bracket.

In another implementation, the magnet has a first magnet and a second magnet. The apparatus also includes a first U-shaped bracket carrying the first magnet and having ends and a second U-shaped bracket carrying the second magnet and having ends. The plate receptacle has a thermal block and a first magnetic permeability material extending from the thermal block and a second magnetic permeability material extending from the thermal block. The thermal block, the first magnetic permeability material, and the second magnetic permeability material defining well receptacles.

In another implementation, the actuator includes a first actuator and a second actuator. The first actuator to move the first U-shaped bracket to allow the first magnetic permeability material to be coupled to and extend between the ends of the first U-shaped bracket. The second actuator is to move the second U-shaped bracket to allow the second magnetic permeability material to be coupled to and extend between the ends of the second U-shaped bracket.

In another implementation, the thermocycler is positioned within the second U-shaped bracket and a stage includes the thermocycler having a thermal block defining first well receptacles and the magnet includes second well receptacles spaced from the first well receptacles.

In another implementation, the magnet has a plurality of ring magnets and each ring magnet surrounds one of the second well receptacles.

In another implementation, the stage is to align the first well receptacles or the second well receptacles with the second well receptacle.

It should be appreciated that all combinations of the foregoing concepts and additional concepts discussed in greater detail below (provided such concepts are not mutually inconsistent) are contemplated as being part of the subject matter disclosed herein and/or may be combined to achieve the particular benefits of a particular aspect described herein. In particular, all combinations of claimed subject matter appearing at the end of this disclosure are contemplated as being part of the subject matter disclosed herein.

### BRIEF DESCRIPTION OF THE DRAWINGS

FIG. 1 illustrates a schematic diagram of an implementation of a system in accordance with the teachings of this disclosure.

FIG. 2 is a cross-sectional view of an implementation of a plate receptacle, a thermocycler, a pair of magnets, and a pair of actuators with the magnets in a first position that can be used to implement the plate receptacle, the thermocycler, the magnet, and the actuator of FIG. 1.

FIG. 3 is another cross-sectional view of the plate receptacle, the thermocycler, the pair of magnets, and the pair of actuators of FIG. 2 with the magnets in a second position.

FIG. 4 is a cross-sectional view of an implementation of a plate receptacle, a thermocycler, a magnet, and an actuator with the magnet in a first position that can be used to implement the plate receptacle, the thermocycler, the magnet, and the actuator of FIG. 1.

FIG. 5 is a cross-sectional view of the plate receptacle, the thermocycler, the magnet, and the actuator of FIG. 4 with the magnet in a second position.

FIG. 6 is a cross-sectional view of an implementation of a plate receptacle, a thermocycler, a magnet, and an actuator with the magnet in a first position that can be used to implement the plate receptacle, the thermocycler, the magnet, and the actuator of FIG. 1.

FIG. 7 is a cross-sectional view of the plate receptacle, the thermocycler, the magnet, and the actuator of FIG. 6 with the magnet in a second position.

FIG. 8 is a cross-sectional view of an implementation of a plate receptacle, a thermocycler, a magnet, and an actuator that can be used to implement the plate receptacle, the thermocycler, the magnet, and the actuator of FIG. 1.

FIG. 9 is a cross-sectional view of an implementation of a plate receptacle, the thermocycler, a first magnet, a second magnet, and an actuator that can be used to implement the plate receptacle, the thermocycler, the magnet, and the actuator of FIG. 1.

FIG. 10 is a cross-sectional view of an implementation of the plate receptacle, the thermocycler, a first magnet, a second magnet, a first actuator, and a second actuator that can be used to implement the plate receptacle, the thermocycler, the magnet, and the actuator of FIG. 1.

FIG. 11 a cross-sectional view of an implementation of another plate receptacle, the thermocycler, the first magnet, the second magnet, the first actuator, and the second actuator that can be used to implement the first plate receptacle, the thermocycler, the magnet, and the actuator of FIG. 1.

FIG. 12 is cross-sectional view of an implementation of a stage including a thermocycler and a magnet and another stage that can be used to implement the thermocycler, and the magnet of FIG. 1 and/or can be used to implement the system of FIG. 1.

FIG. 13 is a cross-sectional view of the stages of FIG. 11 showing the wells of the plate positioned in second well receptacles.

FIG. 14 is cross-sectional view of an implementation of a stage and a coolant system that can be used to implement the thermocycler, and the magnet of FIG. 1 and/or can be used to implement the system of FIG. 1.

### DETAILED DESCRIPTION

Although the following text discloses a detailed description of implementations of methods, apparatuses and/or articles of manufacture, it should be understood that the legal scope of the property right is defined by the words of the claims set forth at the end of this patent. Accordingly, the following detailed description is to be construed as examples only and does not describe every possible implementation, as describing every possible implementation would be impractical, if not impossible. Numerous alternative implementations could be implemented, using either current technology or technology developed after the filing date of this patent. It is envisioned that such alternative implementations would still fall within the scope of the claims.

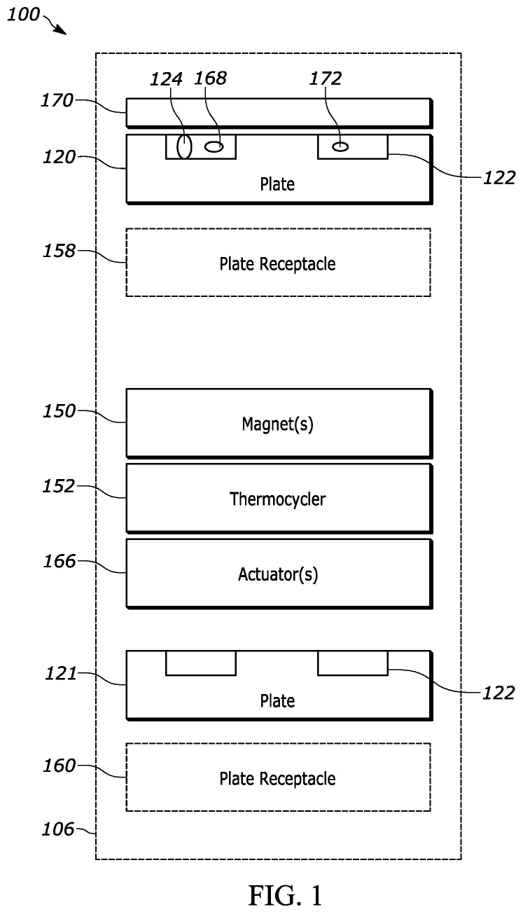

FIG. 1 illustrates a schematic diagram of an implementation of a system 100 in accordance with the teachings of this disclosure. The system 100 may can be used to perform amplification and cleanup operations when preparing DNA libraries for sequencing applications, for example. The system 100 may can thus perform DNA library preparation workflows that include amplification processes, cleanup processes, for example.

The system 100 is shown carrying plates 120, 121 having wells 122 and includes a working area 106 having a magnet 150, a thermocycler 152, a first plate receptacle 158, a second plate receptacle 160, and an actuator 166 in the implementation shown. The magnet 150 may be a permanent magnet or electro-magnetic. The plate receptacles 158, 160 may be referred to as plate stations. Different wells 122 of the first plate 120 may contain different samples 124. The samples 124 may be a biological sample derived from a human, animal, plant, bacteria, or fungi. Other sources of obtaining the biological samples may prove suitable.

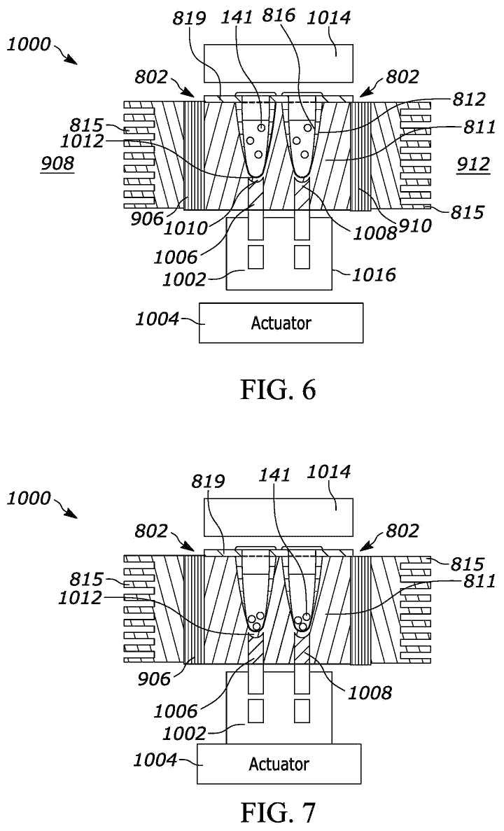

The actuator 166 moves the magnet 150 relative to the first plate receptacle 158 in operation. The actuator 166 can move the magnet 150 between an upward position where the magnet 150 affects any plate positioned on the first plate receptacle 158 and a downward position where the magnet 150 does not affect any plate positioned on the first plate receptacle 158. This may not work as well for FIGS. 6 and 7, but could be applicable to the rest.

The magnet 150 being moved relative to the first plate receptacle 158 and any plate 120, 121 positioned on the first plate receptacle 158 allows less area on the working area 106 to be consumed. The magnet 150 can moreover be moved with relatively higher confidence as compared to an alternative approach to moving one of the plates 120, 121 filled with samples to a separate magnet station. The magnet 150 may be implemented by a second array configuration to strengthen and focus the corresponding magnetic fields.

Reagents 168 and/or samples 124 are dispensed into the well 122 of the first plate 120 and a lid 170 is positioned on the first plate 120 to cover the well 122 of the first plate 120 with the lid 170. The lid 170 may alternatively be movably coupled to the thermocycler 152 (see, FIG. 6, for example). The lid 170 may not be disposable in such implementations.

The thermocycler 152 is aligned with and/or in heat communication with the first plate receptacle 158 and the thermocycler 152 adjusts a temperature of the sample 124 within the well 122 of the first plate 120. Enzymes and reagents 168 amplify the nucleic acid in the sample 124.

The thermocycler 152 can be programed to run a series of temperature adjustments that allow the enzymes and reagents 168 in the well 122 to amplify the nucleic acid of the sample 124 in same well 122. The thermocycler 152 and/or the magnet 150 can, thus, act on a plate 120, 121 received at the first plate receptacle 158. The thermocycler 152 may alternatively be spaced from the magnet 150.

The system 100 may perform the cleanup processes after the amplification processes are performed. Beads 172 may be dispensed into the well 140 of the first plate 120 as part of the cleanup process and a first reagent may be dispensed into the well 122 of the first plate 120. The first reagent may be a bead buffer and the sample 124 may bind to the beads 172 in the presence of the bead buffer.

The actuator 166 moves the magnet 150 toward the first plate receptacle 158 and the magnet 150 draws the beads 172 toward the magnet 150. The beads 172 and the sample 124 bound to the beads 172 may be positioned toward the bottom of the well 122 of the first plate 120 or on a side(s) of the well 122. The actuator 166 may change a position of the magnet 150 relative to the first plate receptacle 158 as a way to adjust a magnetic field strength that acts on the beads 172. A tip of a pipette may easily access the well 122 if the beads 172 are on the side of the well 122. The magnet 150 may cause the beads 172 to be in any position within the well 122, however.

The first reagent may be aspirated from the well 122 of the first plate 120 and dispensed into the first reagent aspirated from the well 122 of the first plate 120 into waste. A second reagent may be dispensed into the well 122 of the first plate 120. The second reagent may be an elution buffer that releases the sample 124 from being bound to the beads 172 and, specifically, releases DNA associated with the sample 124 from being bound to the beads 172. The actuator 166 moves the magnet 150 toward the first plate receptacle 158 to draw the beads 172 toward the magnet 150 and, thus, suspend the second reagent and the sample 124 within the well 122.

The second reagent and the sample 124 may be aspirated from the well 122 of the first plate 120 using a tip of a pipette, for example, and the second reagent and the sample 124 may be dispensed into the well 128 of the second plate 121.

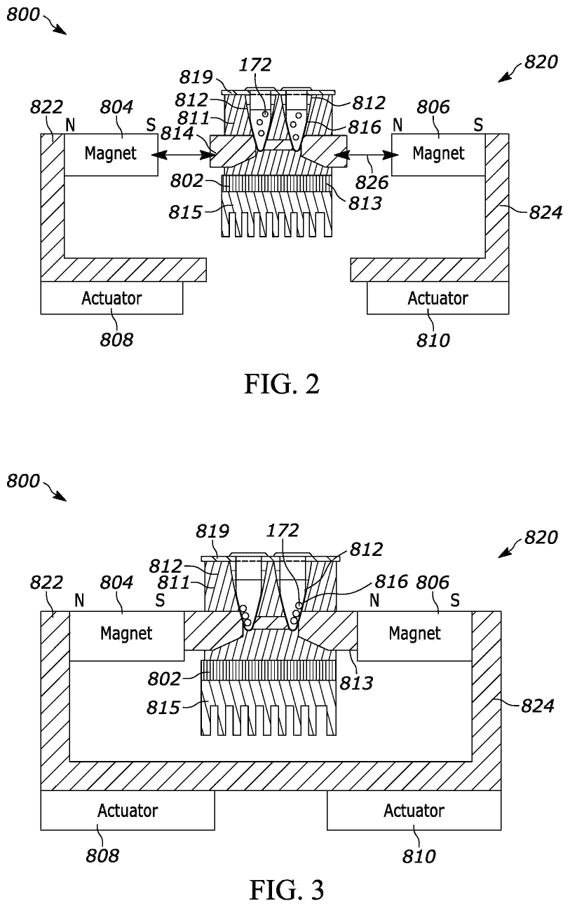

FIG. 2 is a cross-sectional view of an implementation of a plate receptacle 800, a thermocycler 802, a pair of magnets 804, 806, and a pair of actuators 808, 810 with the magnets 804, 806 in a first position that can be used to implement the plate receptacle 158, the thermocycler 152, the magnet 150, and the actuator 166 of FIG. 1. The plate receptacle 800 has a thermal block 811 defining well receptacles 812 and the thermocycler 802 is positioned beneath the well receptacles 812 and is, thus, positioned beneath the plate receptacle 800. The thermal block 811 may be metal and/or a 3D heat pipe. The metal may include aluminum, silver, copper, and/or brass. The thermocycler 802 may additionally or alternatively be a resistive heater.

An insert 814 is positioned within the thermal block 811 and is shown at least partially defining the well receptacles 812 and a heat sink 815 is coupled to the thermocycler 802. The insert 814 may be a ferrous material insert and/or the insert may be operable to transfer a magnetic field from the magnet 804, 806 to the plate receptable 800. The insert 814 extends horizontally through the thermal block 811 in the implementation shown. The thermal block 811 may be made of a metal such as aluminum or silver and the insert 814 may be made of iron. Wells 816 of a plate 819 are shown being received by the well receptacles 812. The plate 819 may be used to implement any one of the first plate 120 or the second plate 121 of FIG. 1.

An actuator assembly 820 is shown having opposing first and second brackets 822, 824 positioned on either side of the plate receptacle 800 in the implementation shown. The first magnet 804 and the first actuator 808 are coupled to the first bracket 822 and the second magnet 806 and the second actuator 810 are coupled to the second bracket 824. While the actuator assembly 820 is shown including the first and second actuators 808, 810, one actuator may alternatively be provided. The first actuator 808 moves the first bracket 822 and the second actuator 810 moves the second bracket 824 relative to the plate receptacle 800 in operation and in directions generally indicated by arrow 826 to allow the first magnet 804 and the second magnet 806 to act on the plate 819. The magnets 804, 806 are shown spaced from the plate 819 and the insert 814 in FIG. 2 and, thus, the beads 172 are suspended within the wells 816. The first position of the actuator assembly 820 shown in FIG. 2 may be referred to as an off position and the second position of the actuator assembly 820 shown in FIG. 3 may be referred to as an on position.

FIG. 3 is another cross-sectional view of the plate receptacle 800, the thermocycler 802, the pair of magnets 804, 806, and the pair of actuators 808, 810 of FIG. 2 with the magnets 804, 806 in a second position. The magnets 804, 806 are shown adjacent the insert 814 and, thus, the magnets 804, 806 magnetize the insert 814 and the beads 172 are drawn toward the corresponding magnets 804, 806. The beads 172 are drawn toward sides of the wells 816 in the implementation shown. The actuator 808, 810 may change a position of the magnets 804, 806 relative to the plate receptacle 800 as a way to adjust a magnetic field strength that acts on the beads 172.

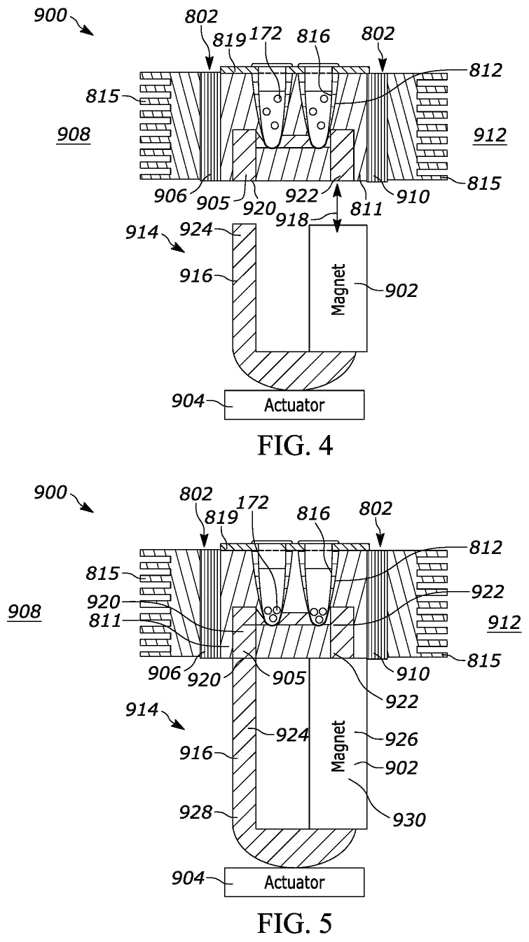

FIG. 4 is a cross-sectional view of an implementation of a plate receptacle 900, a thermocycler 802, a magnet 902, and an actuator 904 with the magnet 902 in a first position that can be used to implement the first plate receptacle 158, the thermocycler 152, the magnet 150, and the actuator 166 of FIG. 1. The plate receptacle 900 has the thermal block 811 defining the well receptacles 812 and an insert 905 is positioned within the thermal block 811. The insert 905 may be a ferrous material insert. The insert 905 is shown at least partially defining the well receptacles 812 and being U-shaped.

The thermocycler 802 of FIG. 4 has a first thermoelectric cooler 906 positioned on a first side 908 of the plate receptacle 900 and a second thermoelectric cooler 910 positioned on a second side 912 of the plate receptacle 900. Heat sinks 815 are coupled to each of the first thermoelectric cooler 906 and the second thermoelectric cooler 910.

An actuator assembly 914 is shown having a bracket 916 positioned beneath the plate receptacle 900 in the implementation shown. The bracket 916 carries the magnet 902 and is shown being a U-shaped bracket. The actuator 904 moves the bracket 916 and the magnet 902 relative to the plate receptacle 900 in directions generally indicated by arrows 918 to allow the magnet 902 to act on the plate 819. The magnet 902 is shown spaced from the plate 819 and the insert 905 in FIG. 4 and, thus, the beads 172 are suspended within the wells 816.

FIG. 5 is a cross-sectional view of the plate receptacle 900, the thermocycler 802, the magnet 902, and the actuator 904 of FIG. 4 with the magnet 902 in a second position. The magnet 902 is shown adjacent the insert 905 and, thus, the magnet 902 magnetizes the insert 905 and the beads 172 are drawn toward the actuator assembly 914 and/or the magnet 902. The insert 905 is U-shaped and has ends 920, 922 and the bracket 916 is also U-shaped and has ends 924, 926 that are positioned adjacent the ends 920, 922 of the insert 905 to allow the magnet 902 to magnetize the insert 905 and, thus, draw the beads 172 toward the insert 905. The bracket 916 is shown having first and second arms 928, 930 and the magnet 902 is shown coupled to the second arm 930. The actuator assembly 914 may alternatively include two of the magnets 902 and each of the first and second arms 928, 930 of the bracket 916 may carry one of the magnets 902.

FIG. 6 is a cross-sectional view of an implementation of a plate receptacle 1000, the thermocycler 802, a magnet 1002, and an actuator 1004 with the magnet 1002 in a first position that can be used to implement the first plate receptacle 158, the thermocycler 152, the magnet 150, and the actuator 166 of FIG. 1. The plate receptacle 1000 has the thermal block 811 defining the well receptacles 812 and inserts 1006, 1008 positioned within the thermal block 811. The inserts 1006, 1008 are positioned beneath the well receptacles 812 and each have a cupped surface 1010 that face the corresponding well receptacle 812. The inserts 1006, 1008 can be referred to as cupped pins.

A portion 1012 of the thermal block 811 is positioned between each of the inserts 1006, 1008 and the corresponding well receptacle 812 for thermal transfer. The portion 1012 may be approximately 1 mm thick. A lid 1014 is positioned over top of the well receptacles 812. The lid 1014 may be operatively coupled to the thermal block 811 and/or hingably coupled to the thermal block 811. The lid 1014 may be a temperature controlled cover that reduces evaporation and improves thermal uniformity. The lid 1014 may be used to implement the lid 170 of FIG. 1.

The magnet 1002 is a switchable magnet 1016 in the implementation shown and the actuator 1004 actuates the switchable magnet 1016 between a non-magnetized position (first position) shown in FIG. 6 and a magnetized position (second position) shown in FIG. 7. The beads 172 are, thus, suspended within the wells 816 in FIG. 6.

FIG. 7 is a cross-sectional view of the plate receptacle 1000, the thermocycler 802, the magnet 1002, and the actuator 1004 of FIG. 6 with the magnet 1002 in a second position. The beads 172 are drawn toward the inserts 1006, 1008 and/or the switchable magnet 1016 in FIG. 7.

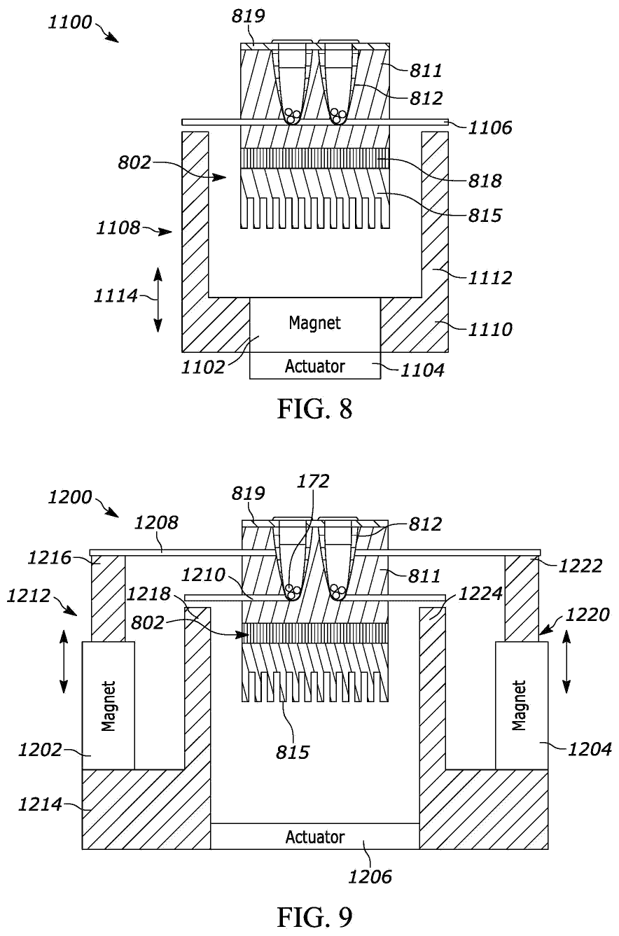

FIG. 8 is a cross-sectional view of an implementation of a plate receptacle 1100, the thermocycler 802, a magnet 1102, and an actuator 1104 can be used to implement the first plate receptacle 158, the thermocycler 152, the magnet 150, and the actuator 166 of FIG. 1. The plate receptacle 1100 has the thermal block 811 defining the well receptacles 812 and a magnetic permeability material 1106 extending from the thermal block 811. The magnetic permeability material 1106 is shown at least partially defining the well receptacles 812. The magnetic permeability material 1106 extends horizontally through the thermal block 811 in the implementation shown. The magnetic permeability material 1106 may include mu-metal, ferritic stainless steel, and/or Metglas.

An actuator assembly 1108 is shown having a bracket 1110 and the magnet 1102 is carried by the bracket 1110. The bracket 1110 is shown as a U-shaped bracket 1112 and the thermocycler 802 is positioned within the U-shaped bracket 1112. The actuator 1104 is coupled to the bracket 1110 and moves the bracket 1110 in operation between in directions generally indicated by arrow 1114 to allow the magnet 1102 to act on the magnetic permeability material 1106 and draw the beads 172 toward the magnetic permeability material 1106.

The well receptacles 812 and the corresponding wells 816 are spaced and/or positioned to allow side access to each of the wells 816 because the plate 819 has two rows of the wells 816 in the implementation shown. The magnetic permeability material 1106 may route and/or shape the magnetic fields as a result of the position of the wells 816. The plate 819 may include additional rows of the wells 816 and the magnetic permeability material may still route and/or shape the magnet field if the wells 816 are spaced a threshold distance apart, for example.

FIG. 9 is a cross-sectional view of an implementation of a plate receptacle 1200, the thermocycler 802, a first magnet 1202, a second magnet 1204, and an actuator 1206 that can be used to implement the first plate receptacle 158, the thermocycler 152, the magnet 150, and the actuator 166 of FIG. 1. The plate receptacle 1200 has the thermal block 811 defining well receptacles 812 and a first magnetic permeability material 1208 extending from the thermal block 811 and a second magnetic permeability material 1210 extending from the thermal block 811. The first magnetic permeability material 1208 and/or the second magnetic permeability material 1210 may be made of a material in which a magnetic fields can induced in when exposed to an external magnetic field. For example, the first magnetic permeability material 1208 and/or the second magnetic permeability material 1210 may include at least one of a ferrous material, Nickel, a mu-metal, or a Cobalt alloy.

The thermal block 811, the first magnetic permeability material 1208, and the second magnetic permeability material 1210 are shown at least partially defining the well receptacles 812. The first magnetic permeability material 1208 and the second magnetic permeability material 1210 extend horizontally through the thermal block 811 and are spaced apart in the implementation shown. The magnetic permeability material 1208, 1210 may be configured to minimize or reduce the time to focus the beads 141 and to achieve a threshold spatial distribution of the beads 141 to minimize or reduce any bead loss in wash operations and minimize or reduce the liquid volume used in an elution step.

An actuator assembly 1212 is shown including a first bracket 1214 carrying the first magnet 1202 and having first and second ends 1216, 1218, and a second bracket 1220 carrying the second magnet 1204 and having first and second ends 1222, 1224. The first bracket 1214 and the second bracket 1220 oppose one another. The thermocycler 802 is positioned between the first bracket 1214 and the second bracket 1220. The actuator 1206 moves the first bracket 1214 and the second bracket 1220 in operation relative to the first magnetic permeability material 1208 and the second magnetic permeability material 1210 to allow the magnets 1202, 1204 to act on the first magnetic permeability material 1208 and the magnets 1202, 1204 to act on the second magnetic permeability material 1210. The magnets 1202, 1204 acting on the first magnetic permeability material 1208 and the second magnetic permeability material 1210 and the first magnetic permeability material 1208 and the second magnetic permeability material 1210 being spaced apart may create a magnetic field distribution that draws the beads 172 toward the bottom of the wells 816.

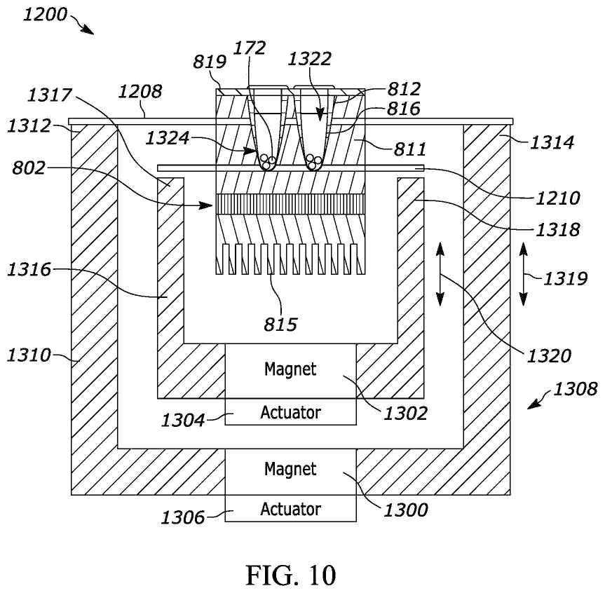

FIG. 10 is a cross-sectional view of an implementation of the plate receptacle 1200, the thermocycler 802, a first magnet 1300, a second magnet 1302, a first actuator 1304, and a second actuator 1306 that can be used to implement the first plate receptacle 158, the thermocycler 152, the magnet 150, and the actuator 166 of FIG. 1. The plate receptacle 1200 of FIG. 9 is substantially the same as the plate receptacle 1200 of FIG. 10.

An actuator assembly 1308 is included, however, that is different from the actuator assembly 1212 of FIG. 8. The actuator assembly 1308 of FIG. 9 is shown including a first U-shaped bracket 1310 carrying the first magnet 1300 and having ends 1312, 1314 and a second U-shaped bracket 1316 carrying the second magnet 1302 and having ends 1317, 1318. The thermocycler 802 is positioned within the second U-shaped bracket 1316.

The ends 1312, 1314 of the first U-shaped bracket 1310 are shown being arranged to magnetize the first magnetic permeability material 1208 and the ends 1317, 1318 of the second U-shaped bracket 1316 are shown being arranged to magnetize the second magnetic permeability material 1210. The first actuator 1304 moves the first U-shaped bracket 1310 to allow the first magnetic permeability material 1208 to be coupled to and extend between the ends 1312, 1314 of the first U-shaped bracket 1310 and the second actuator 1306 moves the second U-shaped bracket 1316 to allow the second magnetic permeability material 1210 to be coupled to and extend between the ends 1317, 1318 of the second U-shaped bracket 1316. The first actuator 1304 can move the first U-shaped bracket 1310 relative to the second U-shaped bracket 1316 in directions generally indicated by arrow 1319 and the second actuator 1306 can move the second U-shaped bracket 1316 relative to the first U-shaped bracket 1310 in directions generally indicated by arrow 1320. The first actuator 1304 and the second actuator 1306 are, thus, independently actuatable.

The first U-shaped bracket 1310 may engage and magnetize the first magnetic permeability material 1208 during first operations to capture the beads 172 around a first position 1322 in the well receptacles 812 and the second U-shaped bracket 1316 may engage and magnetize the second magnetic permeability material 1210 during second operations to capture the beads 172 around a second position 1324 in the well receptacles 812. The first operations may be wash operations and the second operations may be elution operations.

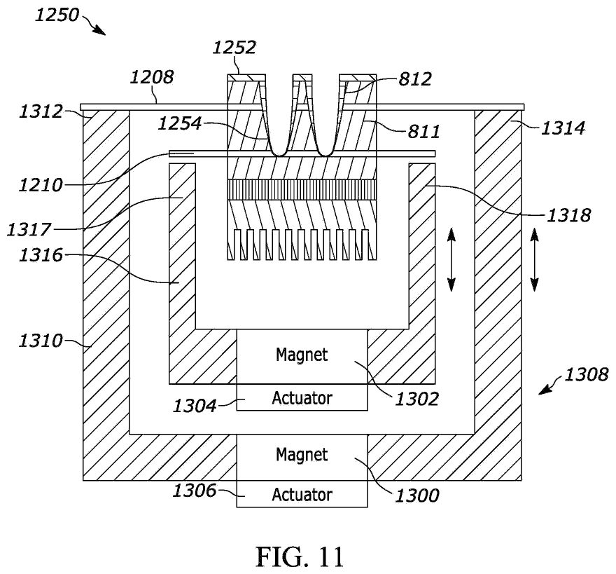

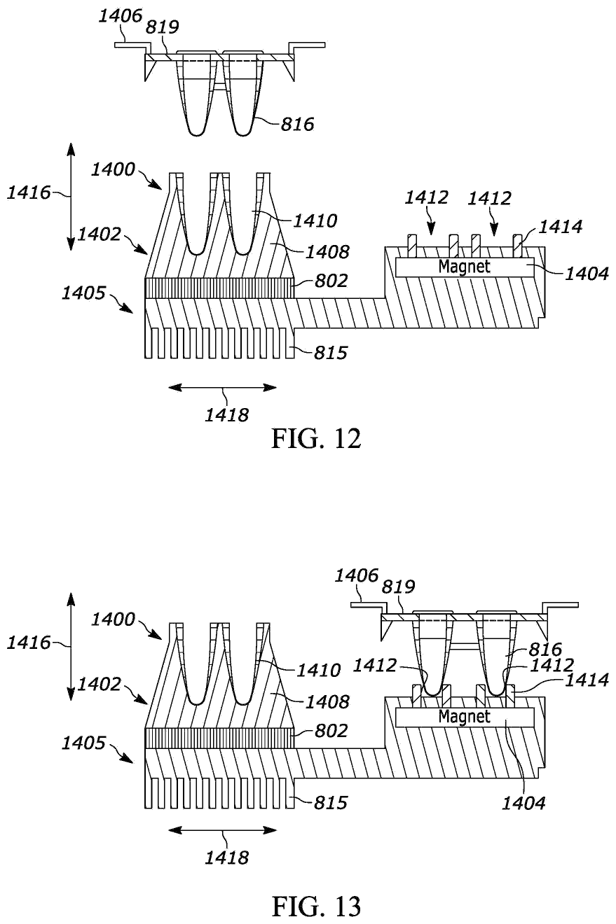

FIG. 11 a cross-sectional view of an implementation of another plate receptacle 1250, the thermocycler 802, the first magnet 1300, the second magnet 1302, the first actuator 1304, and the second actuator 1306 that can be used to implement the first plate receptacle 158, the thermocycler 152, the magnet 150, and the actuator 166 of FIG. 1. The plate receptacle 1250 of FIG. 11 is similar to the plate receptacle 1200 of FIG. 9. The plate receptacle 1250 of FIG. 12, however, includes a plating or a coating 1252 over surfaces 1254 that define the well receptacles 812. The plating or the coating 1252 may reduce exposure of the magnetic permeability materials 1208, 1210 to moisture and/or humidity and, thus, reduce a likelihood that the magnetic permeability materials 1208, 1210 corrode.

FIG. 12 is cross-sectional view of an implementation of a stage 1400 including a thermocycler 1402 and a magnet 1404 and another stage 1406 that can be used to implement the thermocycler 152, and the magnet 150 of FIG. 1 and/or can be used to implement the system 100 of FIG. 1.

The thermocycler 1402 includes a thermal block 1408 defining first well receptacles 1410 and the magnet 1404 has second well receptacles 1412 spaced from the first well receptacles 1410. The magnet 1404 may include ring magnets 1414, where each of the ring magnets 1414 surrounds one of the second well receptacles 1412. The stage 1406 positions and/or holds the plate 819 above the stage 1400 in operation and the stage 1400 aligns the first well receptacles 1410 or the second well receptacles 1412 with the plate 819. The stage 1400 positions the wells 816 of the plate 819 in the first well receptacles 1410 or the second well receptacles 1412 depending on the operations being performed. The stage 1406 can move in directions generally indicated by arrow 1416 and the stage 1400 can move in directions generally indicated by arrow 1418.

FIG. 13 is a cross-sectional view of the stages 1400, 1406 of FIG. 12 showing the wells 816 of the plate 819 positioned in the second well receptacles 1412. The magnet 1404 can act on the wells 816 and/or contents of the wells 816 as a result.

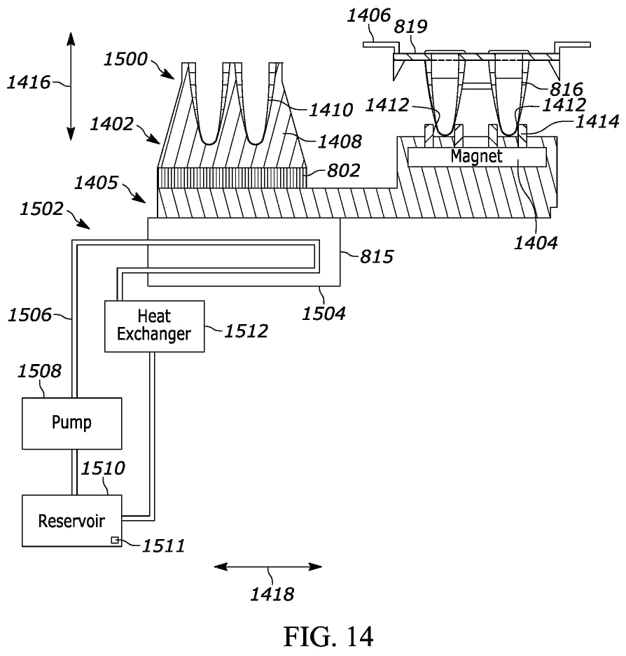

FIG. 14 is cross-sectional view of an implementation of a stage 1500 and a coolant system 1502 that can be used to implement the thermocycler 152, and the magnet 150 of FIG. 1 and/or can be used to implement the system 100 of FIG. 1. The stage 1500 of FIG. 14 is similar to the stage 1400 of FIG. 12 but includes the heat sink 815 that is implemented by a liquid cooled heat sink 1504, however.

The coolant system 1502 is shown including a fluidic line 1506, a pump 1508, a reservoir 1510 containing coolant 1511, and a heat exchanger 1512. The heat sink 1504 may be considered to be part of the coolant system 1502. The fluidic line 1506 passes through the heat sink 1504 in the implementation shown. The heat exchanger 1512 may be referred to as a radiator and the coolant may be a liquid such as water, antifreeze, and/or Ethylene glycol.

The pump 1508 pumps the coolant 1511 from the reservoir 1510 through the fluidic line 1506 and the heat sink 1504 in operation. The coolant 1511 may enter the heat sink 1504 at a first temperature, draw heat from the thermocycler 152 while within a portion of the fluidic line 1506 within the heat sink 1504, and the coolant 1511 may exit the heat sink 1504 at a second temperature. The second temperature may be higher than the first temperature. The coolant 1511 exiting the heat sink 1504 may enter the heat exchanger 1512. The heater changer 1512 may reduce the temperature of the coolant 1511 from the second temperature back to the first temperature or an otherwise reduced temperature. The coolant 1511 entering the reservoir 1510 may be the same or similar to the temperature of the coolant 1511 within the reservoir 1510 and/or not increase the temperature of the coolant 1511 within the reservoir 1510, for example.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

As used herein, an element or step recited in the singular and proceeded with the word “a” or “an” should be understood as not excluding plural of said elements or steps, unless such exclusion is explicitly stated. Furthermore, references to “one implementation” are not intended to be interpreted as excluding the existence of additional implementations that also incorporate the recited features. Moreover, unless explicitly stated to the contrary, implementations “comprising,” “including,” or “having” an element or a plurality of elements having a particular property may include additional elements whether or not they have that property. Moreover, the terms “comprising,” including,” having,” or the like are interchangeably used herein.

The terms “substantially,” “approximately,” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. In one example, these terms include situation where there is no variation—0%.

There may be many other ways to implement the subject technology. Various functions and elements described herein may be partitioned differently from those shown without departing from the scope of the subject technology. Various modifications to these implementations may be readily apparent to those skilled in the art, and generic principles defined herein may be applied to other implementations. Thus, many changes and modifications may be made to the subject technology, by one having ordinary skill in the art, without departing from the scope of the subject technology. For instance, different numbers of a given module or unit may be employed, a different type or types of a given module or unit may be employed, a given module or unit may be added, or a given module or unit may be omitted.

Underlined and/or italicized headings and subheadings are used for convenience only, do not limit the subject technology, and are not referred to in connection with the interpretation of the description of the subject technology. All structural and functional equivalents to the elements of the various implementations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

1. An apparatus, comprising:
a plate receptacle comprising a thermal block and an insert;
a magnet;
a thermocycler in heat communication with the plate receptacle; and
an actuator,
wherein the plate receptacle is to receive a plate having a well and the thermocycler is to adjust a temperature of a sample within the well of the plate,
wherein the actuator is to move the magnet relative to the plate receptacle, and
wherein the insert is operable to transfer a magnetic field from the magnet to the plate receptable.

2. The apparatus of claim 1, wherein the plate receptacle comprises well receptacles and wherein the insert is positioned within the thermal block.

3. The apparatus of claim 2, wherein the insert at least partially defines the well receptacles.

4. The apparatus of claim 1, wherein the insert comprises at least one of a ferrous material, Nickel, a mu-metal, or a Cobalt alloy.

5. The apparatus of claim 1, wherein the insert extends horizontally through the thermal block.

6. The apparatus of claim 1, further comprising a heat sink coupled to the thermocycler.

7. The apparatus of claim 6, wherein the heat sink comprises a liquid cooled heat sink.

8. The apparatus of claim 1, further comprising opposing first and second brackets positioned on either side of the plate receptacle, wherein the magnet comprises a first magnet coupled to the first bracket and a second magnet coupled to the second bracket and wherein the actuator includes a first actuator that moves the first bracket and a second actuator that moves the second bracket relative to the plate receptacle to allow the first magnet and the second magnet to act on the plate receptacle.

9. The apparatus of claim 1, wherein the thermocycler comprises a first thermoelectric cooler positioned on a first side of the plate receptacle and a second thermoelectric cooler positioned on a second side of the plate receptacle.

10. The apparatus of claim 9, further comprising a heatsink coupled to each of the first thermoelectric cooler and the second thermoelectric cooler.

11. The apparatus of claim 1, wherein the magnet comprises a switchable magnet.

12. The apparatus of claim 11, wherein the actuator is to actuate the switchable magnet.

13. The apparatus of claim 1, wherein the plate receptacle comprises a plurality of well receptacles and wherein the insert comprises a plurality of cupped pins, wherein each cupped pin is positioned beneath one of the well receptacles.

14. The apparatus of claim 1, further comprising a U-shaped bracket carrying the magnet and having ends and wherein the insert comprises a magnetic permeability material extending from the thermal block, the thermal block and the magnetic permeability material defining well receptacles.

15. The apparatus of claim 14, wherein the magnetic permeability material comprises at least one of a ferrous material, Nickel, a mu-metal, or a Cobalt alloy.

16. The apparatus of claim 1, wherein the thermocycler is positioned within the U-shaped bracket.

17. The apparatus of claim 1, wherein the magnet comprises a first magnet and a second magnet, further comprising a first bracket carrying the first magnet and having first and second ends and a second bracket carrying the second magnet and having first and second ends, the first bracket and the second bracket opposing one another, wherein the insert comprises a first magnetic permeability material extending from the thermal block and a second magnetic permeability material extending from the thermal block, wherein the thermal block, the first magnetic permeability material, and the second magnetic permeability material defining well receptacles.

18. The apparatus of claim 17, wherein the actuator moves the first bracket and the second bracket to allow the first magnetic permeability material to be coupled to and extend between the first ends of the first bracket and the second bracket and to allow the second magnetic permeability material to be coupled to and extend between the second ends of the first bracket and the second bracket.

19. The apparatus of claim 1, wherein the magnet comprises a first magnet and a second magnet, further comprising a first U-shaped bracket carrying the first magnet and having ends and a second U-shaped bracket carrying the second magnet and having ends, wherein the insert comprises a first magnetic permeability material extending from the thermal block and a second magnetic permeability material extending from the thermal block, wherein the thermal block, the first magnetic permeability material, and the second magnetic permeability material defining well receptacles.

20. The apparatus of claim 19, wherein the actuator comprises a first actuator and a second actuator, wherein the first actuator to move the first U-shaped bracket to allow the first magnetic permeability material to be coupled to and extend between the ends of the first U-shaped bracket, wherein the second actuator is to move the second U-shaped bracket to allow the second magnetic permeability material to be coupled to and extend between the ends of the second U-shaped bracket.

21. The apparatus of claim 1, further comprising a stage comprising the thermocycler comprising the thermal block defining first well receptacles and the magnet comprising second well receptacles spaced from the first well receptacles.

22. The apparatus of claim 21, wherein the magnet comprises a plurality of ring magnets and wherein each ring magnet surrounds one of the second well receptacles.

23. A method, comprising:
adjusting a temperature of a sample within a well of a plate disposed in a plate receptacle comprising a thermal block and an insert using a thermocycler;
dispensing beads into the well of the plate and a first reagent into the well of the plate with the plate disposed in the plate receptacle;
moving the magnet toward the insert to allow the insert to transfer a magnetic field from the magnet to the plate receptable and draw the beads toward the magnet; and
aspirating the first reagent from the well with the plate disposed in the plate receptacle.

24. (canceled)

25. The method of claim 23, further comprising moving the magnet away from the insert.

26. The method of claim 23, further comprising dispensing a second reagent into the well of the first plate.

27. The method of claim 26, further comprising moving the magnet toward the insert to allow the insert to transfer a magnetic field from the magnet to the plate receptable and draw the beads toward the magnet.

28. The method of claim 27, further comprising aspirating the second reagent and the sample from the well of the plate.

29. The method of claim 28, further comprising dispensing the second reagent and the sample into a well of a second plate.

30-57. (canceled)
